(19) United States
(12) Patent Application Publication
(10) Pub. No.: US 2007/0021623 A1
(43) Pub. Date: Jan. 25, 2007

(54) Novel process for preparation of nebivolol intermediates

(75) Inventors: Bandi Parthasaradhi Reddy, Hyderabad (IN); Kura Rathnakar Reddy, Hyderabad (IN); Rapolu Raji Reddy, Hyderabad (IN); Dasari Muralidhara Reddy, Hyderabad (IN); Itiyala Srinivas Reddy, Hyderabad (IN)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Hetero Drugs Limited, Hyderabad, Anhrapradesh (IN)

(21) Appl. No.: 10/568,177

(22) PCT Filed: Aug. 11, 2004
(86) PCT No.: PCT/IN04/00241

§ 371 Date: Feb. 10, 2006

(51) Int. Cl. C07D 405/02

(52) U.S. Cl. 549/403

(57) ABSTRACT

The present invention relates to a process for separation of desired diastereomeric pair from a mixture of diastereomeric pairs thereby obtaining nebivolol intermediates. Thus, the mixture of (+)-[1S*(R*)]-6-fluoro-3,4-di-hydro-α-[[(phenylmethyl)amino]methyl]-2H-1-benzopyran-2-methanol, (+)-[1S*(S*)]-6-fluoro-3,4-dihydro-2-oxi -ranyl-2H-1-benzopyran and ethanol is heated to reflux temperature and stirred for 8 hours at the same temperature to obtain (±)-[2R*[1S*,5S*(S*)]]+[2R*[1S*,5R*(R*)]]-α,α′-[phenylmethyliminobis(methylene)]bis[6-fluoro-3,4-dihydro-2H-1-benzopyran ran-2-methanol]. Then the reaction mass is cooled to 10° C., the pH is adjusted to 2 with HCl gas and stirred for 45 minutes at 25° C. to 30° C. Then the separated solid is filtered and dried to give (+)-[2R*[1S*,5S*(S*)]]-α,α′-[phenylmethyliminobis(methylene)]bis[6-fluoro-3,4-dihydro-2H-1-benzopyran-2-methanol] hydrochloride salt, which can be converted into nebivolol.

### FIELD OF THE INVENTION

The present invention relates to a process for separation of desired diastereomeric pair from a mixture of diastereomeric pairs thereby obtaining nebivolol intermediates.

### BACKGROUND OF THE INVENTION

EP Patent No. 0145067 disclosed 2,2′-iminobisethanol derivatives. The compounds are antihypertensive agents. Among them nebivolol, chemically (+)-[2R*[1S*,5S*(S*)]]-α,α^{l}-[iminobis(methylene)]bis[6-fluoro-3,4-dihydro-2H-1-benzopyran-2-methanol] is the most important antihypertensive agent. Nebivolol is represented by the following structure:

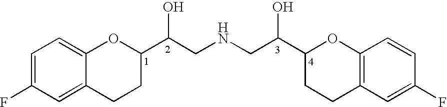

The above structure has four stereogenic centers, which are indicated with No. 1, 2, 3 and 4. Nebivolol is a mixture of equal amounts of 2 enantiomers having respectively the SRRR- and the RSSS-configuration.

Processes for preparations of nebivolol and related compounds were described in EP Patent No. 0145067 and EP Patent No. 0334429. According to the processes described in these patents, chromatographic separations are required for the separation of diastereomeric pairs at the intermediate stage or at the final stage. The chromatographic separations involve additional operations, additional expensive setup adding to the cost of production. U.S. Pat. No. 5,759,580 described the separation of (+)-[2R*[1 S*. 5S*(S*)]]-α,α′-[Iminobis(methylene)]bis[6-fluoro-3,4-dihydro-2H-1-benzopyran-2-methanol] hydrochloride (nebivolol hydrochloride) from the mixture of (+)-[2R*[1 S*, 5S*(S*)]]+[2R*[1S*,5R*(R*)]]-α, α^{l}[iminobis(methylene)]bis[6-fluoro-3,4-dihydro-2H-1-benzopyran-2-methanol]. The yield of the nebivolol hydrochloride is extremely low (6.6%).

We have discovered that when N-protected compound of formula: 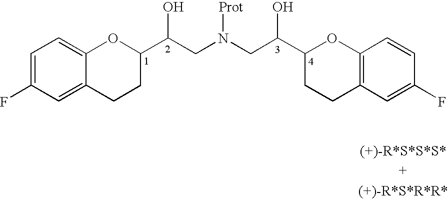
wherein -Prot is a protecting group, is converted into a salt of it, the salts can be subjected to fractional crystallization of the desired diastereomeric pair from the mixture of diastereomeric pairs. The separation of the diastereomers of these N-protected compounds by crystallization is not disclosed in the prior art. The separated diastereomeric pair is a useful intermediate for the preparation of nebivolol.

Fractional crystallization also allows the purification of the N-protected compounds from the reaction mass, thereby avoiding multiple purifications of crude nebivolol.

### DETAILED DESCRIPTION OF THE INVENTION

The present invention provides a novel process for preparing acid additional salts of compounds of formula I: 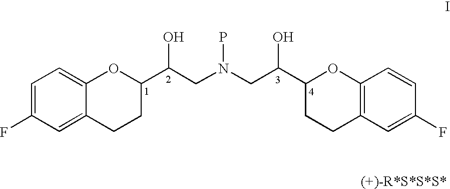
wherein 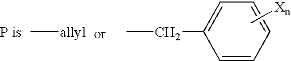
wherein

X each independently is halo, nitro or C₁-C₃ alkyl and n is 0-5; which comprises:
a) treating a mixture containing racemic diastereomers of a compound of formula II: 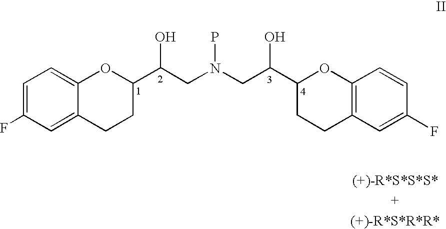
wherein P is as defined in formula I;
with a suitable acid to form the corresponding acid addition salt;
b) subjecting the acid addition salt obtained in step (a) to the fractional crystallization from an alcoholic solvent, ketonic solvent, acetonitrile, dimethylformamide, dimethylsulfoxide and tetrahydrofuran or a mixture thereof to obtain the diastereomeric pair of compounds of formula I.

Stereochemical description describing the configurations at chiral centers used here is in the order (1,2,3 and 4) mentioned in the structure. Thus, for example, the stereochemical description R*S*S*S* shown in the formula I refers to R* configuration at the carbon ‘1’, S* configuration at 2 and so on and R*S*S*S* has the meaning shown below.

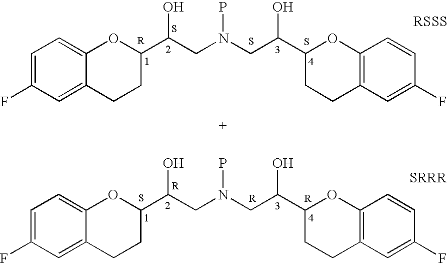

Alcoholic solvents are selected from the group consisting of C₁ to C₅-alcohols. Preferable alcoholic solvents are methanol, ethanol, propanol and isopropyl alcohol.

Ketonic solvents are selected from the group C₃ to C₈-ketones. Preferable ketonic solvents are acetone, methyl isobutyl ketone and methyl tert-butyl ketone.

The acid addition salts are prepared by treating the mixture containing compounds of formula II with the corresponding acids in a solvent by conventional means. The suitable acids are inorganic acids, for example, hydrogen halides, nitric acid, phosphoric acid; and organic acids such as carboxylic acids, sulfonic acids. The examples for carboxylic acids that can be mentioned are acetic acid, propanoic acid, formic acid, hydroxyacetic acid, 2-hydroxy propanoic acid, 2-oxopropanoic acid, propanedioic acid, butanedioic acid, (Z)-2-butenedioic acid, (E)-2-butenedioic acid, 2-hydroxy butanedioic acid. The examples for sulfonic acids that can be mentioned are methane sulfonic acid, toluene sulfonic acid and benzene sulfonic acid.

The step (a) is preferably carried out in an organic solvent. The selection of the solvent is not critical. The solvents may be selected from the group consisting of c-i to C₅ -alcohols, C₃ to C₈-ketones, C₂ to C₈-esters, acetonitrile, tetrahydrofuran, dimethylformamide, dimethylsulfoxide, dioxane, aromatic hydrocarbons, C₁ to C₅-halogenated hydrocarbons and C₂ to C₈-ethers and a mixture thereof. Preferable alcoholic solvents are methanol, ethanol, propanol and isopropyl alcohol; preferable ketonic solvents are acetone, methyl ethyl ketone, methyl isobutyl ketone, methyl tert-butyl ketone and diethyl ketone; preferable ester solvents are ethyl acetate, methyl acetate, isopropyl acetate, tert-butyl methyl acetate and ethyl formate; preferable aromatic hydrocarbon solvents are benzene, toluene and xylene; preferable halogenated hydrocarbon solvents are methylene chloride, chloroform, carbontetrachloride and ethylene dichloride; and preferable ether solvents are tert-butyl methyl ether and diethyl ether. Most preferable solvents are methanol, ethanol, propanol, isopropyl alcohol, acetone, methyl ethyl ketone, methyl isobutyl ketone, methyl tert-butyl ketone, diethyl ketone, acetonitrile, dimethylformamide, dimethylsulfoxide and tetrahydrofuran.

Preferably, fractional crystallization may be carried out in essentially anhydrous conditions. Maintenance of anhydrous conditions during crystallization is not essential but is to avoid the incomplete crystallization of some acid addition salts.

Crystallization may be carried out by commonly known methods such as cooling, addition of an anti-solvent, seeding and partial removal of the solvent or a combination thereof. The fractional crystallization may preferably be carried out at about 0° C. to 45° C. and more preferably at about 0° C. to 35° C.

The acid addition salts of formulas I and II are novel and also form the part of the invention.

The preferred acid addition salts of formula I prepared according to the present invention are hydrogen halides, hydrogen sulfates, sulfates and sulfonic acid salts.

More preferred acid addition salts of formula I are hydrogen halides such as hydrogen chloride, hydrogen iodide and hydrogen bromide, still more preferred salt being hydrogen chloride salt.

Step (a) and (b) can be performed in the same solvent or different solvent. Even though the step (a) and (b) can be performed in different solvents, it is preferred to carry out the salt formation step and fractional crystallization in the same solvent in order to simplify the process.

Acid addition salts can also be prepared from the reaction mass obtained as a part of the synthesis of the compounds of formula II.

The crystalline acid addition salts of the compound of formulas 1 & II are novel.

The more preferred acid addition salts of compound of formula I prepared according to the present invention are hydrogen halide addition salts of formula III:

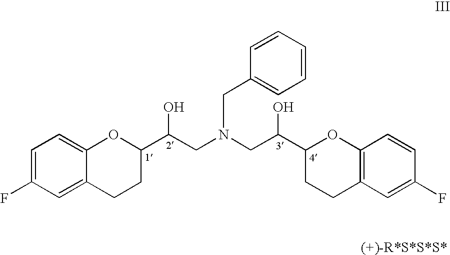

Still more preferred hydrogen halide addition salt of formula III is hydrogen chloride salt.

The process described above may also be used as a purification method for the removal of the undesired diastereomeric pair from the desired diastereomeric pair by basifying the acid addition salt of compound of formula I contaminated with undesired diastereomeric pair and then following the process steps (a) and (b) described above. The purification can be performed till the desired diastereomeric purity level is attained.

The compounds of formula II may be obtained by the methods known in the art. Thus, for example, the compounds of formula II are obtained by the process described in EP Patent No. 0145067 and EP Patent No. 0334429. The patents EP Patent No. 0145067 and EP Patent No. 0334429 are incorporated herein by reference in their entirety.

The acid addition salts of formula I are intermediates for preparing nebivolol and pharmaceutical acceptable salts thereof and can be converted into nebivolol by basifying with a base, removing the protecting group ¹P′ by the processes known in the art and optionally converting nebivolol into a pharmaceutically acceptable salt. The pharmaceutically acceptable salts were described in U.S. Pat. No. 5,759,580 and incorporated herein by reference. The selection of the base is not critical but may be selected from hydroxides, carbonates and bicarbonates of alkaline metals; ammonia and amines. The amine base may be primary amine such as methylamine or ethylamine; secondary amine such as diethylamine or dimethylamine; and tert-amine such as triethylamine, trimethylamine or dimethylaminopyridine. The basification may be carried out in water or in an organic solvent or a mixture thereof. The organic solvents used here may be selected from the group consisting of C₁ to C₅-alcohols, C₃ to C₈-ketones, C₂ to C₈-esters, acetonitrile, tetrahydrofuran, dimsthylformamide, dimethylsulfoxide, dioxane, aromatic hydrocarbons, C₁ to C₅-halogenated hydrocarbons and C₂ to C₈-ethers and a mixture thereof. Preferable alcoholic solvents are methanol, ethanol, propanol and isopropyl alcohol; preferable ketonic solvents are acetone, methyl ethyl ketone, methyl isobutyl ketone, methyl tert-butyl ketone and diethyl ketone; preferable ester solvents are ethyl acetate, methyl acetate, isopropyl acetate, tert-butyl methyl acetate and ethyl formate; preferable aromatic hydrocarbon solvents are benzene, toluene and xylene; preferable halogenated hydrocarbon solvents are methylene chloride, chloroform, carbontetrachloride and ethylene dichloride; and preferable ether solvents are tert-butyl methyl ether and diethyl ether. Most preferable organic solvents are methanol, ethanol, propanol, isopropyl alcohol, acetone, methyl ethyl ketone, methyl isobutyl ketone, methyl tert-butyl ketone, diethyl ketone, acetonitrile, dimethylformamide, dimethylsulfoxide and tetrahydrofuran. Thus, for example, if P is benzyl then catalytic hydrogenation using hydrogenation catalyst such as palladium or platinum on carbon may be used for the de-protection and if P is allyl, then reaction with an appropriate noble metal compound such as PdCl₂ or Rh[P(C₆H₅)3]Cl may be carried out.

The hydrogenation is carried out in a solvent. The selection of the solvent is not critical and may be selected from the group consisting of c-i to C₅-alcohols, C₃^{′ to C}₈-ketones, C₂ to C₈-esters, acetonitrile, tetrahydrofuran, dimethyiformamide, dimethylsulfoxide, dioxane, aromatic hydrocarbons, C₁ to C₅-halogenated hydrocarbons and C₂ to C₈-ethers and a mixture thereof. Preferable alcoholic solvents are methanol, ethanol, propanol and isopropyl alcohol; preferable ketonic solvents are acetone, methyl ethyl ketone, methyl isobutyl ketone, methyl tert-butyl ketone and diethyl ketone; preferable ester solvents are ethyl acetate, methyl acetate, isopropyl acetate, tert-butyl methyl acetate and ethyl formate; preferable aromatic hydrocarbon solvents are benzene, toluene and xylene; preferable halogenated hydrocarbon solvents are methylene chloride, chloroform, carbontetrachloride and ethylene dichloride; and preferable ether solvents are tert-butyl methyl ether and diethyl ether. Most preferable organic solvents are methanol, ethanol, propanol, isopropyl alcohol, acetone, methyl ethyl ketone, methyl isobutyl ketone, methyl tert-butyl ketone, diethyl ketone, acetonitrile, dimethyiformamide, dimethylsulfoxide and tetrahydrofuran.

‘Essentially anhydrous condition” refers to the water content less than 10%, preferably less than 5% and more preferably less than 2% of the total mass by weight.

In a preferred process (+)-[1S*(R*)]- (or (+)-[1 S*(S*)])-6-fluoro-3,4-dihydro-α-[[(phenylmethyl)amino]methyl]-2H-1-benzopyran-2-methanol is reacted with (+)-[1S*(S*)]- (or (+)[1S*(R*)])-6-fluoro-3,4-dihydro-2-oxiranyl-2H-i-benzopyran in an C₁ to C₅-alcohol or C₃ to C₈-ketone solvent to obtain (+)-[2R*[1S*,5S*(S*)]]+[2R*[1S*,5R*(R*)]]-α, α^{I}-[phenylmethyliminobis(methylene)] bis[6-fluoro-3,4-dihydro-2H-1-benzopyran-2-methanol], hydrogen halide, sulfate or hydrogen sulfate is added to the reaction mass, crystallization is performed at about 0° C. to 45° C., the separated solid is filtered to obtain the corresponding salt of (+)-[2R*[1S*,5S*(S*)]]-α, α′-[phenylmethyliminobis(methylene)]bis[6-fluoro-3,4-dihydro-2H-1-benzopyran-2-methanol], the salt is basified in a solvent to obtain (+)-[2R*[1S*, 5S*(S*)]]-a,a′-[phenylmethyliminobis(methylene)bis[6-fluoro-3,4-dihydro-2H-1-benzopyran-2-methanol] free base and the free base is subjected to catalytic hydrogenation with hydrogen using palladium on carbon as catalyst.

The addition of hydrohalide may be performed by passing hydrogen halide gas, e.g., HCl (g) to the reaction mass or adding hydrogen halide dissolved in a solvent to the reaction mass.

Unless otherwise specified, the alkyl portion of the C₁ to C₅-alcohol, C₃ to C₈-ketone, C₂ to C₈-ester, Ci to C₅-halogenated hydrocarbon and C₂ to C₈-ether used can be straight or branch, unsubstituted or substituted with for example alkoxy, halogen, nitro, cyano or hydroxy groups.

The invention will now be further described by the following examples, which are illustrative rather than limiting.

### EXAMPLE 1

The solution of benzyl amine (14.89 gm) in ethanol (90 ml) is added to a mixture of (+)-[1S*(R*)]-6-fluoro-3,4-dihydro-2-oxiranyl-2H-1-benzopyran (9 gm) and ethanol (90 ml) drop wise at reflux temperature for 15 minutes. The temperature of the reaction mixture is raised to reflux and maintained for 5 hours at reflux temperature. Then ethanol is distilled off under vacuum at 50° C. To this residue diisopropyl ether (50 ml) added and stirred for 30 minutes at 0-5° C. Then the separated solid is filtered, washed with chilled diisopropylether and dried to give 8.5 gm of (+)-[1S*(R*)]-6-fluoro-3,4-dihydro-α-[[(phenylmethyl)amino]methyl]-2H-1-benzopyran-2-methanol (HPLC purity: 97%).

### EXAMPLE 2

The mixture of (+)-[1S*(R*)]-6-fluoro-3,4-dihydro-α-[[(phenylmethyl) amino]methyl]-2H-1-benzopyran-2-methanol (100 gm, obtained in example 1), (±)-[1S*(S*)]-6-fluoro-3,4-dihydro-2-oxiranyl-2H-1-benzopyran (90 gm) and ethanol (2000 ml) is heated to reflux temperature and stirred for 8 hours at the same temperature to obtain (+)-[2R*[1S*, 5S*(S*)]]+[2R*[1S*,5R*(R*)]]- α,oc′-[phenylmethyliminobis(methylene)]bis[6-fluoro-3,4-dihydro-2H-1-benzopyran-2-methanol] ((±)-[2R*[1S*, 5S*(S*)]] to (+)-[2R*[1 S*,5R*(R*)]] ratio is 1: 1.1). Then the reaction mass is cooled to 10° C., the pH is adjusted to 2 with HCl gas and stirred for 45 minutes at 25° C. to 30° C. Then the separated solid is filtered and dried to give 80.7 gm of (+)-[2R*[1 S*, 5S*(S*)]]-α,α′-[phenylmethyliminobis (methylene)]bis[6-fluoro-3,4-dihydro-2H-1-benzopyran-2-methanol] hydrochloride salt (HPLC purity 99.0%; (±)-[2R*[1 S*, 5S*(S*)]] to (+)-[2R*[1S*,5R*(R*)]] ratio is 99.4:0.6).

### EXAMPLE 3

The mixture of 10% NaHCO₃ solution (800 ml) and (+)-[2R*[1S*, 5S*(S*)]]-α, α′-[phenylmethyliminobis(methylene)]bis[6-fluoro-3,4-dihydro-2H-1-benzopyran-2-methanol] hydrochloride salt (80.7 gm) obtained above is stirred for 15 minutes and then extracted twice with ethyl acetate (1600 ml). Then the organic layer is washed with water (800 ml) and 20% sodium chloride solution (400 ml) and then distilled off the solvent to give 38.4 gm of (+)-[2R*[1 S*, 5S*(S*)]]-α, α′-[phenylmethyliminobis(methylene)]bis[6-fluoro-3,4-dihydro-2H-1-benzopyran-2-methanol] residue.

The mixture of (+)-[2R*[1S*, 5S*(S*)]]-α,α′-[phenylmethyliminobis (methylene)]bis[6-fluoro-3,4-dihydro-2H-1-benzopyran-2-methanol] residue (38.4 gm, obtained above, 10% palladium on charcoal (10 gm) and ethanol (1300 ml) is taken into a hydrogenation flask and subjected to hydrogenation under a hydrogen gas pressure<of 2.5 kg/cm²for 3 hours. Then the reaction mixture is filtered on hi-flo and washed with ethanol. The solvent is distilled off, acetonitrile (200 ml) is added and stirred for 10 minutes. Then the separated solid is filtered and washed with acetonitrile to give 10 gm of (+)-[2R*[1 SV 5S*(S*)]]-α,α′-[iminobis(methylene)]bis[6-fluoro-3,4-dihydro-2H-1-benzopyran-2-methanol] (Nebivolol) (HPLC purity: 99.3%).

### EXAMPLE 4

Nebivolol (10 gm) obtained in example 3 is dissolved in the mixture of methylene dichloride (150 ml) and ethanol (100 ml) at 45° C. and then cooled to 10° C. Then pH of the solution is adjusted to 2 with HCl gas, stirred for 10 minutes and distilled the solvent. Then the solid is filtered and washed with acetone to give 7 gm of Nebivolol hydrochloride salt (HPLC purity: 99.8%).

1. A process for preparing acid additional salts of compounds of formula I: 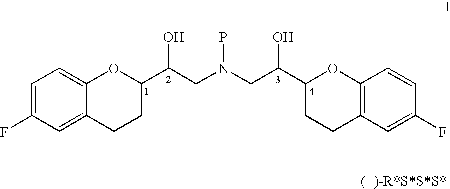
wherein 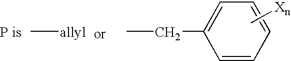
wherein
X each independently is halo, nitro or C₁-C₃ alkyl and n is 0-5;
which comprises:
a) treating a mixture containing racemic diastereomers of a compound of formula II: 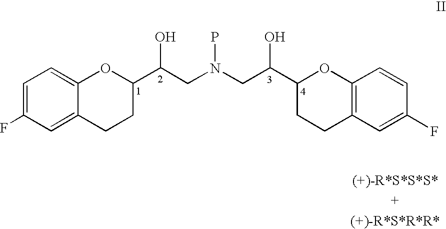
wherein P is as defined in formula I;
with a suitable acid to form the corresponding acid addition salt;
b) subjecting the acid addition salt obtained in step (a) to the fractional crystallization from an alcoholic solvent, ketonic solvent, acetonitrile, dimethylformamide, dimethylsulfoxide and tetrahydrofuran or mixture there of to obtain the diastereomeric pair of compound of formula I.

2. The process according to claim 1, wherein the alcoholic solvent is selected from the group consisting of C₁ to C₅-alcohols.

3. The process according to claim 2, wherein the alcoholic solvent is selected from methanol, ethanol, propanol and isopropyl alcohol.

4. The process according to claim 3, wherein the alcoholic solvent is ethanol.

5. The process according to claim 1, wherein the ketonic solvent is selected from the group consisting of C₃ to C₈-ketones.

6. The process according to claim 5, wherein the ketonic solvent is selected from acetone, methyl isobutyl ketone and methyl tert-butyl ketone.

7. The process according to claim 6, wherein the ketonic solvent is acetone.

8. The process according to claim 1, wherein the acid addition salt is prepared by treating the mixture containing compounds of formula II with the corresponding acid in a solvent.

9. The process according to claim 8, wherein the acid is inorganic acid or an organic acid.

10. The process according to claim 9, wherein the inorganic acid is a hydrogen halide, nitric acid or phosphoric acid.

11. The process according to claim 10, wherein the hydrogen halide is hydrogen chloride.

12. The process according to claim 9, wherein the organic acid is a carboxylic acid or a sulfonic acid.

13. The process according to claim 12, wherein the carboxylic acid is selected from acetic acid, propanoic acid, formic acid, hydroxyacetic acid, 2-hydroxy propanoic acid, 2-oxopropanoic acid, propane dioic acid, butanedioic acid, (Z)-2-butenedioic acid, (E)-2-butenedioic acid and 2-hydroxy butanedioic acid.

14. The process according to claim 12, wherein the sulfonic acid is selected from methane sulfonic acid, toluene sulfonic acid and benzene sulfonic acid.

15. The process according to claim 8, wherein the solvent is an organic solvent.

16. The process according to claim 15, wherein the organic solvent is selected from the group consisting of C₁ to C₅-alcohols, C₃ to C₈-ketones, C₂ to C₈-esters, acetonitrile, tetrahydrofuran, dimethylformamide, dimethylsulfoxide, dioxane, aromatic hydrocarbons, C₁ to C₅-halogenated hydrocarbons and C₂ to C₈-ethers and mixtures thereof.

17. The process according to claim 16, wherein the alcoholic solvents are methanol, ethanol, propanol and isopropyl alcohol; ketonic solvents are acetone, methyl ethyl ketone, methyl isobutyl ketone, methyl tert-butyl ketone and diethyl ketone; ester solvents are ethyl acetate, methyl acetate, isopropyl acetate, tert-butyl methyl acetate and ethyl formate; the aromatic hydrocarbon solvents are benzene, toluene and xylene; the halogenated hydrocarbon solvents are methylene chloride, chloroform, carbontetrachloride and ethylene dichloride; and the ether solvents are tert-butyl methyl ether and diethyl ether.

18. The process according to claim 16, wherein the organic solvent is selected from methanol, ethanol, propanol, isopropyl alcohol, acetone, methyl ethyl ketone, methyl isobutyl ketone, methyl tert-butyl ketone, diethyl ketone, acetonitrile, dimethylformamide, dimethylsulfoxide and tetrahydrofuran.

19. The process according to claim 18, wherein the organic solvent is methanol, ethanol or acetone.

20. The process according to claim 1, wherein the fractional crystallization is carried out in anhydrous condition.

21. The process according to claims 1, wherein the crystallization is carried out by cooling, addition of anti-solvents, seeding or partial removal of the solvents or combinations thereof.

22. The process according to claim 21, wherein the crystallization is carried out at about 0° C. to 45° C.

23. The process according to claim 22, wherein the crystallization is carried out at about 0° C. to 35° C.

24. The process according to claim 1, wherein the steps (a) and (b) are performed in the same solvent or different solvent.

25. The process according to claim 24, wherein the steps (a) and (b) are performed in different solvents.

26. The process according to claim 1, wherein the acid addition salts of formula I are prepared from a reaction mass obtained as a part of the synthesis of the compounds of formula II.

27. The process according to claims 1, wherein the acid addition salts of compound of formula I prepared according to the present invention are hydrogen halide addition salts of formula III:

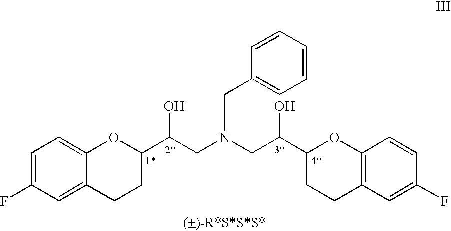

28. The process according to claim 27, wherein the hydrogen halide salt of formula III is hydrogen chloride salt.

29. A process for the preparation of nebivolol or a pharmaceutically acceptable salt thereof, which comprises the steps of:
a) reacting 6-fluoro-3,4-dihydro-α-[[(phenylmethyl)amino]methyl]-2H-1-benzopyran-2-methanol with 6-fluoro-3,4-dihydro-2-oxiranyl-2H-1-benzopyran in an C₁ to C₅-alcohol or C₃ to C₈-ketone solvent to produce (+)-[2R*[1S*,5S*(S*)]]+[2R*[1S*,5R*(R*)]]-α,α′-[phenylmethyliminobis(methylene)]bis[6-fluoro-3,4-dihydro-2H-1-benzopyran-2-methanol];
b) treating (+)-[2R*[1S*,5S*(S*)]]+[2R*[1S*,5R*(R*)]]-α,α′-[phenylmethylimino bis(methylene)]bis[6-fluoro-3,4-dihydro-2H-1-benzopyran-2-methanol] produced in step (a) with hydrogen halide, sulfate or hydrogen sulfate; subjecting to fractional crystallization at about 0° C. to 45° C. and filtering the separated solid to produce corresponding salt of (+)-[2R*[1S*,5S*(S*)]]-α,α′-[phenylmethyliminobis (methylene)] bis[6-fluoro-3,4-dihydro-2H-1-benzopyran -2-methanol];
c) basifying the salt of (+)-[2R*[1S*,5S*(S*)]]-α,α′-[phenylmethylimino bis(methylene)] bis[6-fluoro-3,4-dihydro-2H-1-benzopyran-2-methanol] produced in step(b) in a solvent to produce (+)-[2R*[1S*, 5S*(S*)]]-α,α′-[phenylmethyliminobis(methylene)] bis[6-fluoro-3,4-dihydro-2H-1-benzopyran-2-methanol] free base; and
d) subjecting (+)-[2R*[1S*,5S*(S*)]]-α,α′-[phenylmethyliminobis(methylene)]bis [6-fluoro-3,4-dihydro-2H-1-benzopyran-2-methanol] free base to catalytic hydrogenation with hydrogen using palladium on carbon as a catalyst to obtain nebivolol and optionally converting to the pharmaceutically acceptable salt.

30. The process according to claim 29, wherein the alcoholic solvent is selected from the group consisting of C₁ to C₅-alcohols.

31. The process according to claim 30, wherein the alcoholic solvent is selected from methanol, ethanol, propanol and isopropyl alcohol.

32. The process according to claim 31, wherein the alcoholic solvent is ethanol.

33. The process according to claim 29, wherein the ketonic solvent is selected from the group consisting of C₃ to C₈-ketones.

34. The process according to claim 33, wherein the ketonic solvent is selected from acetone, methyl isobutyl ketone and methyl tert-butyl ketone.

35. The process according to claim 34, wherein the ketonic solvent is acetone.

36. The process according to claim 29, wherein the hydrogen halide is hydrogen chloride.

37. The process according to claim 36, wherein the treatment in step (b) is carried out by passing hydrogen chloride gas to the mass containing (+) -[2R*[1S*, 5S*(S*)]]+[2R*[1S*,5R*(R*)]]-α,α′-[phenylmethyliminobis(methylene)]bis[6-fluoro-3,4-dihydro-2H-1-benzopyran-2-methanol] (or) by adding a solution of hydrogen chloride in a solvent to the mass containing (+)-[2R*[1S*,5S*(S*)]]+[2R* [1S*,5R*(R*)]]-α,α′-[phenylmethyliminobis(methyl ene)]bis[6-fluoro-3,4-dihydro-2H-1-benzopyran-2-methanol].

38. Acid addition salts of formulas I and II: 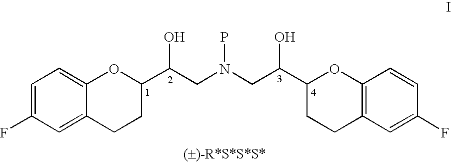
wherein 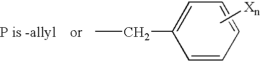
wherein X each independently is halo, nitro or C₁-C₃ alkyl and n is 0-5; 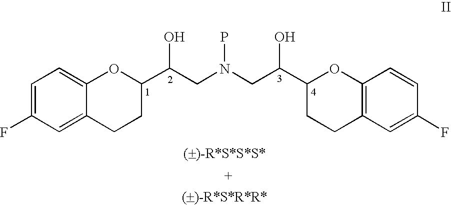
wherein P is as defined in formula I.

39. The acid addition salts of claim 38, wherein the said salts are hydrogen halides, hydrogen sulfates, sulfates and sulfonic acid salts.

40. The acid addition salts of claim 39, wherein the said salts are hydrogen halides.

41. The acid addition salts of claim 40, wherein the said salts are hydrogen chloride, hydrogen iodide and hydrogen bromide.

42. The acid addition salts of claim 41, wherein the said salt is hydrogen chloride.

43. The process according to claim 20, wherein the crystallization is carried out by cooling, addition of anti-solvents, seeding or partial removal of the solvents or combination thereof.

44. The process according to claim 26, wherein the acid addition salts of compound of formula I prepared according to the present invention are hydrogen halide addition salts of formula III: [chemical structure]
